(19) United States
(12) Patent Application Publication
(10) Pub. No.: US 2009/0186359 A1
(43) Pub. Date: Jul. 23, 2009

(54) DETECTING PROSTATE CANCER

(76) Inventors: Abhijit Mazumder, Basking Ridge, NJ (US); Tatiana I. Vener, Stirling, NJ (US); Carlo C. Derecho, Lakehurst, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/355,328

(22) Filed: Jan. 16, 2009

Related U.S. Application Data
(60) Provisional application No. 61/022,650, filed on Jan. 22, 2008.

(51) Int. Cl. C12Q 1/68

(52) U.S. Cl. 435/6

(57) ABSTRACT

An assay for detecting prostate cancer includes reagents for detecting the methylation of GSTP1 and HIC-1 genes.

### BACKGROUND OF THE INVENTION

This invention relates to the interrogation of methylated genes in concert with other diagnostic 5 methods and kits for use with these methods.

Epigenetic changes (alterations in gene expression that do not involve alterations in DNA nucleotide sequences) are primarily comprised of modifications in DNA methylation and remodeling of chromatin. Alterations in DNA methylation have been documented in a wide range of tumors and genes. Esteller et al. (2001); Bastian et al. (2004); and Esteller (2005). The extent of methylation at a particular CpG site can vary across patient samples. Jeronimo et al. (2001); and Pao et al (2001).

A number of potential methylation markers have recently been disclosed. Glutathione S-transferases (GSTs) are exemplary proteins in which the methylation status of the genes that express them can have important prognostic and diagnostic value for prostate cancer. The proteins catalyze intracellular detoxification reactions, including the inactivation of electrophilic carcinogens, by conjugating chemically-reactive electrophiles to glutathione. (Pickett et al. (1989); Coles et al. (1990); and Rushmore et al. (1993). Human GSTs, encoded by several different genes at different loci, have been classified into four families referred to as alpha, mu, pi, and theta. Mannervik et al. (1992). Decreased GSTP1 expression resulting from epigenetic changes is often related to prostate and hepatic cancers.

Computational approaches (Das et al. (2006)) and bisulfite sequencing (Chan et al. (2005)) indicate that multiple sites within a CpG island can be methylated and that the extent of methylation can vary across these sites. For example, in oral cancer, differences in the degree of methylation of individual CpG sites were noted for p16, E-cadherin, cyclin A1, and cytoglobin. Shaw et al. (2006). In prostate and bladder tumors, the endothelin receptor B displayed hotspots for methylation. (Pao et al. (2001). In colorectal and gastric cancer, methylation of the edge of the CpG island of the death-associated protein kinase gene was detected in virtually every sample, in contrast to the more central regions. Satoh et al. (2002). The differential distribution of methylation is found the RASSF1A CpG island in breast cancer and methylation may progressively spread from the first exon into the promoter area. Yan et al. (2003); and Strunnikova et al. (2005). RASSF2 has frequent methylation at the 5′ and 3′ edges of the CpG island, with less frequent methylation near the transcription start site. Endoh et al. (2005).

In endometrial carcinoma four GSTP1 designs showed sensitivities between 14% and 24% but the sample sizes were too small to determine if these differences were real. (Chan et al. 2005). Two assay designs increase sensitivity of detection of prostate carcinoma (Nakayama et al. (2003)); however, both designs shared the same reverse primer so there was considerable overlap in the regions interrogated. Differences exist in the percent methylation for different CpG sequences for p16, E-cadherin, cyclin A1, and cytoglobin. Shaw et al. (2006). Differential methylation levels at CpG sites exist in breast cancer. Yan et al. (2003).

An inverse correlation exists between tumor MLH1 RNA expression and MLH1 DNA methylation. Yu et al. (2006). Methylation-positive samples exhibited lower levels of RNA expression of the DAPK gene in lung cancer cell lines. Toyooka et al. (2003). However, those studies examined only one site of methylation so correlations with RNA expression at multiple locations in a CpG island could not be determined. The core region surrounding the transcription start site is an informative surrogate for promoter methylation. Eckhardt et al. (2006).

In squamous cell carcinoma of the esophagus, methylation at individual genes increased in frequency from normal to invasive cancer. (Guo et al. 2006). Methylation of TMS1(p=0.002), DcR1 (p=−0.01), DcR2 (p=0.03), and CRBP1 (p=0.03) correlate with Gleason score and methylation of CRBP1 correlates with higher stage (p=0.0002) and methylation of Reprimo (p=0.02) and TMS1 (p=0.006) correlated with higher (>8 ng/ml) PSA levels. Suzuki et al. (2006). Methylation status was correlated with the extent of myometrial invasion in endometrial carcinoma. A significantly (p=0.04) higher frequency of ASC methylation in the tumor-adjacent, normal tissue for patients was associated with biochemical recurrence, suggesting a correlation with aggressive disease. Chan et al. (2005). RARb2, PTGS2, and EDNRB may have prognostic value in patients undergoing radical prostatectomy. Bastian et al. (2007).

Methylation-specific PCR (MSP) assays have been performed at multiple sites of two genes known to be methylated in prostate cancer, GSTP1 and RARb2. Lee et al. (1994); Harden et al. (2003); Jeronimo et al. (2004); and Nakayama et al. (2001). It has been reported that •HIC-1 and EDNRB are prostate tissue specific markers. Bastian et al. (2004).

Sampling and sample preparation are important factors in epigenetic testing. Every sample source has its issues. Even biopsy samples taken directly from the affected tissue are known to present the possibility of false negative results due to uneven distribution of affected cells. Urine is a desirable sample because it can be obtained less invasively than many other potential samples. The number and concentration of prostate cancer cells shed into urine can be extremely variable depending on a host of factors such as when the urine is collected, whether it is collected pursuant to prostate massage, and the presence and effect of nucleases and reagents and methods for minimizing their effect.

While some have proposed prostate cancer testing on urine samples, actually producing such a test has proven difficult. First, it is presumed that the basis for such a test is the shedding of cancer cells from the tumor or lesion into the urinary system. Little is actually known about this process. It also seems likely that analyte concentrations could vary much more dramatically than in other samples such as tissue biopsy and even serum samples depending on a wide range of physiological and environmental factors such as the degree to which the patient is hydrated. The stability of the analyte in the matrix is also not well understood in light of the presence of nucleases and a wide variety of other substances that can affect nucleic acids. Sample preparation for a number of other urine assays use spun down samples referred to as sediments. Whether this makes sense for methylation markers cannot be supposed a priori.

Preparation of the patient and pretreatment options are also not well understood. Digital rectal examinations (DRE) are standard diagnostic procedures for determining prostate health in which the physician notes anatomical abnormalities. In the past the outcome of the DRE would be used to determine whether a biopsy or other diagnostic or therapeutic procedure would be necessary. Whether and to what extent procedures such as the DRE or related prostate massage causes cells to slough so that they would then be detected in the subsequent diagnostic procedure was unclear. Procedurally, DRE and digital rectal massage and the time in which they are performed can differ greatly further adding to the list of unknowns in this area.

### SUMMARY OF THE INVENTION

The present invention encompasses methods, kits and reagents for use therein for determining the presence of prostate cancer.

In another aspect of the invention, a method for characterizing prostate cancer in a patient comprises assaying GSTP1 methylation, one or more control genes, and the HIC1 gene. A normalized value of GSTP1 is determined by comparison of the GSTP1 methylation assay value to that of the HIC1 methylation assay value. The assay is considered positive for prostate cancer if the normalized methylation assay value exceeds a pre-determined value and is considered negative for prostate cancer if the pre-determined value is not exceeded.

### DETAILED DESCRIPTION OF THE INVENTION

The modification of nucleic acid sequences having the potential to express proteins, peptides, or mRNA (such sequences referred to as “genes”) within the genome has been shown, by itself, to be determinative of whether a protein, peptide, or mRNA is expressed in a given cell. Whether or not a given gene capable of expressing proteins, peptides, or mRNA does so and to what extent such expression occurs, if at all, is determined by a variety of complex factors. Irrespective of difficulties in understanding and assessing these factors, assaying gene expression or modification patterns can provide useful information about the occurrence of important events such as tumorogenesis, metastasis, apoptosis, and other clinically relevant phenomena. Relative indications of the degree to which genes are active or inactive can be found in gene expression or modification profiles.

A sample can be any biological fluid, cell, tissue, organ or portion thereof that contains genomic DNA suitable for methylation detection. A test sample can include or be suspected to include a neoplastic cell, such as a cell from the colon, rectum, breast, ovary, prostate, kidney, lung, blood, brain or other organ or tissue that contains or is suspected to contain a neoplastic cell. The term includes samples present in an individual as well as samples obtained or derived from the individual. For example, a sample can be a histologic section of a specimen obtained by biopsy, or cells that are placed in or adapted to tissue culture. A sample further can be a subcellular fraction or extract, or a crude or substantially pure nucleic acid molecule or protein preparation. A reference sample can be used to establish a reference level and, accordingly, can be derived from the source tissue that meets having the particular phenotypic characteristics to which the test sample is to be compared.

A sample for determining gene modification profiles can be obtained by any method known in the art. Samples can be obtained according to standard techniques from all types of biological sources that are usual sources of genomic DNA including, but not limited to cells or cellular components which contain DNA, cell lines, biopsies, bodily fluids such as blood, sputum, stool, urine, cerebrospinal fluid, ejaculate, tissue embedded in paraffin such as tissue from eyes, intestine, kidney, brain, heart, prostate, lung, breast or liver, histological object slides, and all possible combinations thereof. A suitable biological sample can be sourced and acquired subsequent to the formulation of the diagnostic aim of the marker. A sample can be derived from a population of cells or from a tissue that is predicted to be afflicted with or phenotypic of the condition. The genomic DNA can be derived from a high-quality source such that the sample contains only the tissue type of interest, minimum contamination and minimum DNA fragmentation.

Sample preparation requires the collection of patient samples. Patient samples used in the inventive method are those that are suspected of containing diseased cells such as epithelial cells taken from the primary tumor in a colon sample or from surgical margins. Laser Capture Microdissection (LCM) technology is one way to select the cells to be studied, minimizing variability caused by cell type heterogeneity. Consequently, moderate or small changes in gene expression between normal and cancerous cells can be readily detected. Samples can also comprise circulating epithelial cells extracted from peripheral blood. These can be obtained according to a number of methods but the most preferred method is the magnetic separation technique described in U.S. Pat. No. 6,136,182. Once the sample containing the cells of interest has been obtained, DNA is extracted and amplified and a cytosine methylation profile is obtained, for genes in the appropriate portfolios.

DNA methylation and methods related thereto are discussed for instance in US patent publication numbers 20020197639, 20030022215, 20030032026, 20030082600, 20030087258, 20030096289, 20030129620, 20030148290, 20030157510, 20030170684, 20030215842, 20030224040, 20030232351, 20040023279, 20040038245, 20040048275, 20040072197, 20040086944, 20040101843, 20040115663, 20040132048, 20040137474, 20040146866, 20040146868, 20040152080, 20040171118, 20040203048, 20040241704, 20040248090, 20040248120, 20040265814, 20050009059, 20050019762, 20050026183, 20050053937, 20050064428, 20050069879, 20050079527, 20050089870, 20050130172, 20050153296, 20050196792, 20050208491, 20050208538, 20050214812, 20050233340, 20050239101, 20050260630, 20050266458, 20050287553 and U.S. Pat. Nos. 5,786,146, 6,214,556, 6,251,594, 6,331,393 and 6,335,165.

DNA modification kits are commercially available, they convert purified genomic DNA with unmethylated cytosines into genomic lacking unmethylated cytosines but with additional uracils. The treatment is a two-step chemical process consisting a deamination reaction facilitated by bisulfite and a desulfonation step facilitated by sodium hydroxide. Typically the deamination reaction is performed as a liquid and is terminated by incubation on ice followed by adding column binding buffer. Following solid phase binding and washing the DNA is eluted and the desulfonation reaction is performed in a liquid. Adding ethanol terminates the reaction and the modified DNA is cleaned up by precipitation. However, both commercially available kits (Zymo and Chemicon) perform the desulfonation reaction while the DNA is bound on the column and washing the column terminates the reaction. The treated DNA is eluted from the column ready for MSP assay.

The step of isolating DNA may be conducted in accordance with standard protocols. The DNA may be isolated from any suitable body sample, such as cells from tissue (fresh or fixed samples), blood (including serum and plasma), semen, urine, lymph or bone marrow. For some types of body samples, particularly fluid samples such as blood, semen, urine and lymph, it may be preferred to firstly subject the sample to a process to enrich the concentration of a certain cell type (e.g. prostate cells). One suitable process for enrichment involves the separation of required cells through the use of cell-specific antibodies coupled to magnetic beads and a magnetic cell separation device.

Prior to the amplifying step, the isolated DNA is preferably treated such that unmethylated cytosines are converted to uracil or another nucleotide capable of forming a base pair with adenine while methylated cytosines are unchanged or are converted to a nucleotide capable of forming a base pair with guanine.

Preferably, following treatment and amplification of the isolated DNA, a test is performed to verify that unmethylated cytosines have been efficiently converted to uracil or another nucleotide capable of forming a base pair with adenine, and that methylated cytosines have remained unchanged or efficiently converted to another nucleotide capable of forming a base pair with guanine.

Preferably, the treatment of the isolated DNA involves reacting the isolated DNA with bisulphite in accordance with standard protocols. In bisulphite treatment, unmethylated cytosines are converted to uracil whereas methylated cytosines will be unchanged. Verification that unmethylated cytosines have been converted to uracil and that methylated cystosines have remained unchanged may be achieved by; (i) restricting an aliquot of the treated and amplified DNA with a suitable restriction enzyme which recognize a restriction site generated by or resistant to the bisulphite treatment, and (ii) assessing the restriction fragment pattern by electrophoresis. Alternatively, verification may be achieved by differential hybridization using specific oligonucleotides targeted to regions of the treated DNA where unmethylated cytosines would have been converted to uracil and methylated cytosines would have remained unchanged. The amplifying step may involve polymerase chain reaction (PCR) amplification, ligase chain reaction amplification and others. Warnecke et al. (1997).

Preferably, the amplifying step is conducted in accordance with standard protocols for PCR amplification, in which case, the reactants will typically be suitable primers, dNTPs and a thermostable DNA polymerase, and the conditions will be cycles of varying temperatures and durations to effect alternating denaturation of strand duplexes, annealing of primers (e.g. under high stringency conditions) and subsequent DNA synthesis.

To achieve selective PCR amplification with bisulphite-treated DNA, primers and conditions may be used to discriminate between a target region including a site or sites of abnormal cytosine methylation and a target region where there is no site or sites of abnormal cytosine methylation. Thus, for amplification only of a target region where the said site or sites at which abnormal cytosine methylation occurs is/are methylated, the primers used to anneal to the bisulphite-treated DNA (i.e. reverse primers) may include a guanine nucleotide at a site at which it will form a base pair with a methylated cytosine. Such primers will form a mismatch if the target region in the isolated DNA has unmethylated cytosine nucleotide (which would have been converted to uracil by the bisulphite treatment) at the site or sites at which abnormal cytosine methylation occurs. The primers used for annealing to the opposite strand (i.e. the forward primers) may include a cytosine nucleotide at any site corresponding to site of methylated cytosine in the bisulphite-treated DNA.

The step of amplifying is used to amplify a target region within the GST-Pi gene and/or its regulatory flanking sequences. The regulatory flanking sequences may be regarded as the flanking sequences 5′ and 3′ of the GST-Pi gene which include the elements that regulate, either alone or in combination with another like element, expression of the GST-Pi gene.

Sites of abnormal cytosine methylation can be detected for the purposes of diagnosing or prognosing a disease or condition by methods which do not involve selective amplification. For instance, oligonucleotide/polynucleotide probes could be designed for use in hybridization studies (e.g. Southern blotting) with bisulphite-treated DNA which, under appropriate conditions of stringency, selectively hybridize only to DNA which includes a site or sites of abnormal methylation of cytosine. Alternatively, an appropriately selected informative restriction enzyme can be used to produce restriction fragment patterns that distinguish between DNA which does and does not include a site or sites of abnormal methylation of cytosine.

The method of the invention can also include contacting a nucleic acid-containing specimen with an agent that modifies unmethylated cytosine; amplifying the CpG containing nucleic acid in the specimen by means of CpG-specific oligonucleotide primers; and detecting the methylated nucleic acid. The preferred modification is the 15 conversion of unmethylated cytosines to another nucleotide that will distinguish the unmethylated from the methylated cytosine. Preferably, the agent modifies unmethylated cytosine to uracil and is sodium bisulfite, however, other agents that modify unmethylated cytosine, but not methylated cytosine can also be used. Sodium bisulfite (NaHSO₃) modification is most preferred and reacts readily with the 5,6-double bond of cytosine, but poorly with methylated cytosine. Cytosine reacts with the bisulfite ion to form a sulfonated cytosine reaction intermediate susceptible to deamination, giving rise to a sulfonated uracil. The sulfonate group can be removed under alkaline conditions, resulting in the formation of uracil. Uracil is recognized as a thymine by Taq polymerase and therefore upon PCR, the resultant product contains cytosine only at the position where 5-methylcytosine occurs in the starting template. Scorpion reporters and reagents and other detection systems similarly distinguish modified from unmodified species treated in this manner.

The primers used in the invention for amplification of a CpG-containing nucleic acid in the specimen, after modification (e.g., with bisulfite), specifically distinguish between untreated DNA, methylated, and non-methylated DNA. In methylation specific PCR (MSPCR), primers or priming sequences for the non-methylated DNA preferably have a T in the 3′ CG pair to distinguish it from the C retained in methylated DNA, and the complement is designed for the antisense primer. MSP primers or priming sequences for non-methylated DNA usually contain relatively few Cs or Gs in the sequence since the Cs will be absent in the sense primer and the Gs absent in the antisense primer (C becomes modified to U (uracil) which is amplified as T (thymidine) in the amplification product).

The primers of the invention are oligonucleotides of sufficient length and appropriate sequence so as to provide specific initiation of polymerization on a significant number of nucleic acids in the polymorphic locus. When exposed to appropriate probes or reporters, the sequences that are amplified reveal methylation status and thus diagnostic information. Preferred primers are most preferably eight or more deoxyribonucleotides or ribonucleotides capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The priming segment of the primer or priming sequence is preferably single stranded for maximum efficiency in amplification, but may be double stranded. If double stranded, the primer is first treated to separate its strands before being used to prepare extension products. The primer must be sufficiently long to prime the synthesis of extension products in the presence of the inducing agent for polymerization. The exact length of primer will depend on factors such as temperature, buffer, cations, and nucleotide composition. The oligonucleotide primers most preferably contain about 12-20 nucleotides although they may contain more or fewer nucleotides, preferably according to well known design guidelines or rules. Primers are designed to be substantially complementary to each strand of the genomic locus to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with the 5′ and 3′ flanking sequence(s) to hybridize and permit amplification of the genomic locus. The primers are employed in the amplification process. That is, reactions (preferably, an enzymatic chain reaction) that produce greater quantities of target locus relative to the number of reaction steps involved. In a most preferred embodiment, the reaction produces exponentially greater quantities of the target locus. Reactions such as these include the PCR reaction. Typically, one primer is complementary to the negative (−) strand of the locus and the other is complementary to the positive (+) strand. Annealing the primers to denatured nucleic acid followed by extension with an enzyme, such as the large fragment of DNA Polymerase I (Klenow) and nucleotides, results in newly synthesized + and − strands containing the target locus sequence. The product of the chain reaction is a discrete nucleic acid duplex with termini corresponding to the ends of the specific primers employed.

The primers may be prepared using any suitable method, such as conventional phosphotriester and phosphodiester methods including automated methods. In one such automated embodiment, diethylphosphoramidites are used as starting materials and may be synthesized as described by Beaucage et al. (1981). A method for synthesizing oligonucleotides on a modified solid support is described in U.S. Pat. No. 4,458,066.

Any nucleic acid specimen taken from urine or urethral wash, in purified or non-purified form, can be utilized as the starting nucleic acid or acids, provided it contains, or is suspected of containing, the specific nucleic acid sequence containing the target locus (e.g., CpG). Thus, the process may employ, for example, DNA or RNA, including messenger RNA. The DNA or RNA may be single stranded or double stranded. In the event that RNA is to be used as a template, enzymes, and/or conditions optimal for reverse transcribing the template to DNA would be utilized. In addition, a DNA-RNA hybrid containing one strand of each may be utilized. A mixture of nucleic acids may also be employed, or the nucleic acids produced in a previous amplification reaction herein, using the same or different primers may be so utilized. The specific nucleic acid sequence to be amplified, i.e., the target locus, may be a fraction of a larger molecule or can be present initially as a discrete molecule so that the specific sequence constitutes the entire nucleic acid.

If the extracted sample is impure, it may be treated before amplification with an amount of a reagent effective to open the cells, fluids, tissues, or animal cell membranes of the sample, and to expose and/or separate the strand(s) of the nucleic acid(s). This lysing and nucleic acid denaturing step to expose and separate the strands will allow amplification to occur much more readily.

Where the target nucleic acid sequence of the sample contains two strands, it is necessary to separate the strands of the nucleic acid before it can be used as the template. Strand separation can be effected either as a separate step or simultaneously with the synthesis of the primer extension products. This strand separation can be accomplished using various suitable denaturing conditions, including physical, chemical or enzymatic means. One physical method of separating nucleic acid strands involves heating the nucleic acid until it is denatured. Typical heat denaturation may involve temperatures ranging from about 80 to 105° C. for up to 10 minutes. Strand separation may also be induced by an enzyme from the class of enzymes known as helicases or by the enzyme RecA, which has helicase activity, and in the presence of riboATP, is known to denature DNA. Reaction conditions that are suitable for strand separation of nucleic acids using helicases are described by Kuhn Hoffmann-Berling (1978). Techniques for using RecA are reviewed in Radding (1982). Refinements of these techniques are now also well known.

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, preferably at a pH of 7-9, most preferably about 8. A molar excess (for genomic nucleic acid, usually about 10₈:1, primer template) of the two oligonucleotide primers is preferably added to the buffer containing the separated template strands. The amount of complementary strand may not be known if the process of the invention is used for diagnostic applications, so the amount of primer relative to the amount of complementary strand cannot always be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90-100° C. for up to 10 minutes, preferably from 1 to 4minutes. After this heating period, the solution is allowed to cool to room temperature, which is preferable for the primer hybridization. To the cooled mixture is added an appropriate agent for effecting the primer extension reaction (the “agent for polymerization”), and the reaction is allowed to occur under conditions known in the art. The agent for polymerization may also be added together with the other reagents if it is heat stable. This synthesis (or amplification) reaction may occur at room temperature up to a temperature at which the agent for polymerization no longer functions. The agent for polymerization may be any compound or system that will function to accomplish the synthesis of primer extension products, preferably enzymes. Suitable enzymes for this purpose include, for example, E. coli DNA polymerase 1, Klenow fragment of E. coli DNA polymerase I, T4 DNA polymerase, other available DNA polymerases, polymerase mutants, reverse transcriptase, and other enzymes, including heat-stable enzymes (e.g., those enzymes which perform primer extension after being subjected to temperatures sufficiently elevated to cause denaturation). A preferred agent is Taq polymerase. Suitable enzymes will facilitate combination of the nucleotides in the proper manner to form the primer extension products complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

Most preferably, the method of amplifying is by PCR. Alternative methods of amplification can also be employed as long as the methylated and non-methylated loci amplified by PCR using the primers of the invention is similarly amplified by the alternative means. In one such most preferred embodiment, the assay is conducted as a nested PCR. In nested PCR methods, two or more staged polymerase chain reactions are undertaken. In a first-stage polymerase chain reaction, a pair of outer oligonucleotide primers, consisting of an upper and a lower primer that flank a particular first target nucleotide sequence in the 5′ and 3′ position, respectively, are used to amplify that first sequence. In subsequent stages, a second set of inner or nested oligonucleotide primers, also consisting of an upper and a lower primer, are used to amplify a smaller second target nucleotide sequence that is contained within the first target nucleotide sequence.

The upper and lower inner primers flank the second target nucleotide sequence in the 5′ and 3′ positions, respectively. Flanking primers are complementary to segments on the 3′-end portions of the double-stranded target nucleotide sequence that is amplified during the PCR process. The first nucleotide sequence within the region of the gene targeted for amplification in the first-stage polymerase chain reaction is flanked by an upper primer in the 5′ upstream position and a lower primer in the 3′ downstream position. The first targeted nucleotide sequence, and hence the amplification product of the first-stage polymerase chain reaction, has a predicted base-pair length, which is determined by the base-pair distance between the 5′ upstream and 3′ downstream hybridization positions of the upper and lower primers, respectively, of the outer primer pair.

At the end of the first-stage polymerase chain reaction, an aliquot of the resulting mixture is carried over into a second-stage polymerase chain reaction. This is preferably conducted within a sealed or closed vessel automatically such as with the “SMART CAP” device from Cepheid. In this second-stage reaction, the products of the first-stage reaction are combined with specific inner or nested primers. These inner primers are derived from nucleotide sequences within the first targeted nucleotide sequence and flank a second, smaller targeted nucleotide sequence contained within the first targeted nucleotide sequence. This mixture is subjected to initial denaturation, annealing, and extension steps, followed by thermocycling as before to allow for repeated denaturation, annealing, and extension or replication of the second targeted nucleotide sequence. This second targeted nucleotide sequence is flanked by an upper primer in the 5′ upstream position and a lower primer in the 3′ downstream position. The second targeted nucleotide sequence, and hence the amplification product of the second-stage PCR, also has a predicted base-pair length, which is determined by the base-pair distance between the 5′ upstream and 3′ downstream hybridization positions of the upper and lower primers, respectively, of the inner primer pair.

The amplified products are preferably identified as methylated or non-methylated with a probe or reporter specific to the product as described in U.S. Pat. No. 4,683,195. Advances in the field of probes and reporters for detecting polynucleotides are well known to those skilled in the art.

Optionally, the methylation pattern of the nucleic acid can be confirmed by other techniques such as restriction enzyme digestion and Southern blot analysis. Examples of methylation sensitive restriction endonucleases which can be used to detect 5′CpG methylation include SmaI, SacII, EagI, MspI, HpaII, BstUI and BssHII.

In another aspect of the invention a methylation ratio is used. This can be done by establishing a ratio between the amount of amplified methylated species of Marker attained and the amount of amplified reference Marker or non-methylated Marker region amplified. This is best done using quantitative real-time PCR. Ratios above an established or predetermined cutoff or threshold are considered hypermethylated and indicative of having a proliferative disorder such as cancer (prostate cancer in the case of GSTP1). Cutoffs are established according to known methods in which such methods are used for at least two sets of samples: those with known diseased conditions and those with known normal conditions. The reference Markers of the invention can also be used as internal controls. The reference Marker is preferably a gene that is constitutively expressed in the cells of the samples such as Beta Actin.

Established or predetermined values (cutoff or threshold values) are also established and used in methods according to the invention in which a ratio is not used. In this case, the cutoff value is established with respect to the amount or degree of methylation relative to some baseline value such as the amount or degree of methylation in normal samples or in samples in which the cancer is clinically insignificant (is known not to progress to clinically relevant states or is not aggressive). These cutoffs are established according to well-known methods as in the case of their use in methods based on a methylation ratio.

Since a decreased level of transcription of the gene associated with the Marker is often the result of hypermethylation of the polynucleotide sequence and/or particular elements of the expression control sequences (e.g., the promoter sequence), primers prepared to match those sequences were prepared. Accordingly, the invention provides methods of detecting or diagnosing a cell proliferative disorder by detecting methylation of particular areas, preferably, within the expression control or promoter region of the Markers. Probes useful for detecting methylation of these areas are useful in such diagnostic or prognostic methods.

The kits of the invention can be configured with a variety of components provided that they all contain at least one primer or probe or a detection molecule (e.g., Scorpion reporter). In one embodiment, the kit includes reagents for amplifying and detecting hypermethylated Marker segments. Optionally, the kit includes sample preparation reagents and /or articles (e.g., tubes) to extract nucleic acids from samples.

In a preferred kit, reagents necessary for one-tube MSP are included such as, a corresponding PCR primer set, a thermostable DNA polymerase, such as Taq polymerase, and a suitable detection reagent(s) such as hydrolysis probe or molecular beacon. In optionally preferred kits, detection reagents are Scorpion reporters or reagents. A single dye primer or a fluorescent dye specific to double-stranded DNA such as ethidium bromide can also be used. The primers are preferably in quantities that yield high concentrations. Additional materials in the kit may include: suitable reaction tubes or vials, a barrier composition, typically a wax bead, optionally including magnesium; necessary buffers and reagents such as dNTPs; control nucleic acid(s) and/or any additional buffers, compounds, co-factors, ionic constituents, proteins and enzymes, polymers, and the like that may be used in MSP reactions. Optionally, the kits include nucleic acid extraction reagents and materials.

A Biomarker is any indicia of an indicated Marker nucleic acid/protein. Nucleic acids can be any known in the art including, without limitation, nuclear, mitochondrial (homeoplasmy, heteroplasmy), viral, bacterial, fungal, mycoplasmal, etc. The indicia can be direct or indirect and measure over- or under-expression of the gene given the physiologic parameters and in comparison to an internal control, placebo, normal tissue or another carcinoma. Biomarkers include, without limitation, nucleic acids and proteins (both over and under-expression and direct and indirect). Using nucleic acids as Biomarkers can include any method known in the art including, without limitation, measuring DNA amplification, deletion, insertion, duplication, RNA, microRNA (miRNA), loss of heterozygosity (LOH), single nucleotide polymorphisms (SNPs, Brookes (1999)), copy number polymorphisms (CNPs) either directly or upon genome amplification, microsatellite DNA, epigenetic changes such as DNA hypo- or hyper-methylation and FISH. Using proteins as Biomarkers includes any method known in the art including, without limitation, measuring amount, activity, modifications such as glycosylation, phosphorylation, ADP-ribosylation, ubiquitination, etc., or imunohistochemistry (IHC) and turnover. Other Biomarkers include imaging, molecular profiling, cell count and apoptosis Markers.

A Marker gene corresponds to the sequence designated by a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein, it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

The inventive methods, compositions, articles, and kits of described and claimed in this specification include one or more Marker genes. “Marker” or “Marker gene” is used throughout this specification to refer to genes and gene expression products that correspond with any gene the over- or under-expression of which is associated with an indication or tissue type.

Preferred methods for establishing gene expression profiles include determining the amount of RNA that is produced by a gene that can code for a protein or peptide. This is accomplished by reverse transcriptase PCR (RT-PCR), competitive RT-PCR, real time RT-PCR, differential display RT-PCR, Northern Blot analysis and other related tests. While it is possible to conduct these techniques using individual PCR reactions, it is best to amplify complementary DNA (cDNA) or complementary RNA (cRNA) produced from mRNA and analyze it via microarray. A number of different array configurations and methods for their production are known to those of skill in the art and are described in for instance, U.S. Pat. Nos. 5,445,934; 5,532,128; 5,556,752; 5,242,974; 5,384,261; 5,405,783 ; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637.

Microarray technology allows for the measurement of the steady-state mRNA level of thousands of genes simultaneously thereby presenting a powerful tool for identifying effects such as the onset, arrest, or modulation of uncontrolled cell proliferation. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. Nos. 6,271,002; 6,218,122; 6,218,114; and 6,004,755.

Analysis of the expression levels is conducted by comparing such signal intensities. This is best done by generating a ratio matrix of the expression intensities of genes in a test sample versus those in a control sample. For instance, the gene expression intensities from a diseased tissue can be compared with the expression intensities generated from benign or normal tissue of the same type. A ratio of these expression intensities indicates the fold-change in gene expression between the test and control samples.

The selection can be based on statistical tests that produce ranked lists related to the evidence of significance for each gene's differential expression between factors related to the tumor's original site of origin. Examples of such tests include ANOVA and Kruskal-Wallis. The rankings can be used as weightings in a model designed to interpret the summation of such weights, up to a cutoff, as the preponderance of evidence in favor of one class over another. Previous evidence as described in the literature may also be used to adjust the weightings.

A preferred embodiment is to normalize each measurement by identifying a stable control set and scaling this set to zero variance across all samples. This control set is defined as any single endogenous transcript or set of endogenous transcripts affected by systematic error in the assay, and not known to change independently of this error. All Markers are adjusted by the sample specific factor that generates zero variance for any descriptive statistic of the control set, such as mean or median, or for a direct measurement. Alternatively, if the premise of variation of controls related only to systematic error is not true, yet the resulting classification error is less when normalization is performed, the control set will still be used as stated. Non-endogenous spike controls could also be helpful, but are not preferred.

Gene expression profiles can be displayed in a number of ways. The most common is to arrange raw fluorescence intensities or ratio matrix into a graphical dendogram where columns indicate test samples and rows indicate genes. The data are arranged so genes that have similar expression profiles are proximal to each other. The expression ratio for each gene is visualized as a color. For example, a ratio less than one (down-regulation) appears in the blue portion of the spectrum while a ratio greater than one (up-regulation) appears in the red portion of the spectrum. Commercially available computer software programs are available to display such data including “Genespring” (Silicon Genetics, Inc.) and “Discovery” and “Infer” (Partek, Inc.)

In the case of measuring protein levels to determine gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent or other detectable reagents to facilitate detection. Methods of detection include, without limitation, enzyme-linked immunosorbent assay (ELISA) and immunoblot techniques.

Modulated genes used in the methods of the invention are described in the Examples. The genes that are differentially expressed are either up regulated or down regulated in patients with carcinoma of a particular origin relative to those with carcinomas from different origins. Up regulation and down regulation are relative terms meaning that a detectable difference (beyond the contribution of noise in the system used to measure it) is found in the amount of expression of the genes relative to some baseline. In this case, the baseline is determined based on the algorithm. The genes of interest in the diseased cells are then either up regulated or down regulated relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis may include the determination of disease/status issues such as determining the likelihood of relapse, type of therapy and therapy monitoring. In therapy monitoring, clinical judgments are made regarding the effect of a given course of therapy by comparing the expression of genes over time to determine whether the gene expression profiles have changed or are changing to patterns more consistent with normal tissue.

Genes can be grouped so that information obtained about the set of genes in the group provides a sound basis for making a clinically relevant judgment such as a diagnosis, prognosis, or treatment choice. These sets of genes make up the portfolios of the invention. As with most diagnostic Markers, it is often desirable to use the fewest number of Markers sufficient to make a correct medical judgment. This prevents a delay in treatment pending further analysis as well unproductive use of time and resources.

One method of establishing gene expression portfolios is through the use of optimization algorithms such as the mean variance algorithm widely used in establishing stock portfolios. This method is described in detail in 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is preferred. Markowitz (1952). Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional Markers such as serum protein Markers (e.g., Cancer Antigen 27.29 (“CA 27.29”)). A range of such Markers exists including such analytes as CA 27.29. In one such method, blood is periodically taken from a treated patient and then subjected to an enzyme immunoassay for one of the serum Markers described above. When the concentration of the Marker suggests the return of tumors or failure of therapy, a sample source amenable to gene expression analysis is taken. Where a suspicious mass exists, a fine needle aspirate (FNA) is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

Methods of isolating nucleic acid and protein are well known in the art. See e.g. U.S. Pat. No. 6,992,182 incorporated by reference herein in its entirety and the discussion of RNA isolation at the Ambion website on the World Wide Web of the Internet, and US 20070054287.

DNA analysis can be any known in the art including, without limitation, methylation, de-methylation, karyotyping, ploidy (aneuploidy, polyploidy), DNA integrity (assessed through gels or spectrophotometry), translocations, mutations, gene fusions, activation—de-activation, single nucleotide polymorphisms (SNPs), copy number or whole genome amplification to detect genetic makeup. RNA analysis includes any known in the art including, without limitation, q-RT-PCR, miRNA or post-transcription modifications. Protein analysis includes any known in the art including, without limitation, antibody detection, post-translation modifications or turnover. The proteins can be cell surface markers, preferably epithelial, endothelial, viral or cell type. The Biomarker can be related to viral/bacterial infection, insult or antigen expression.

Kits made according to the invention include formatted assays for determining the gene expression profiles. These can include all or some of the materials needed to conduct the assays such as reagents and instructions and a medium through which Biomarkers are assayed.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting cancer.

The urine-based assay of this invention is preferably conducted on patients who have had a PSA assay with ambiguous or difficult to determine results (most preferably 2.5-4.0 ng/ml). A negative result using the assay of this invention (in the absence of other clinical indicia) can spare the patient of more invasive testing such as with a biopsy procedure. Thus, viewed most inclusively, one method according to the invention involves first conducting a PSA test in a patient and then conducting the assay described more fully below on those patients having a PSA level assayed at 2.5-4.0 ng/ml.

The assays of the invention detect hypermethylation of nucleic acids that correspond to particular genes whose methylation status correlates with prostate cancer. A nucleic acid corresponds to a gene whose methylation status correlates with prostate cancer when methylation status of such a gene provides information about prostate cancer and the sequence is a coding portion of the gene or its complement, a representative portion of the gene or its complement, a promoter or regulatory sequence for the gene or its complement, a sequence that indicates the presence of the gene or its complement, or the full length sequence of the gene or its complement. Such nucleic acids are referred to as Markers in this specification. Markers correspond to the following genes only: GSTP1, APC, RARβ2, HINC1. Preferably the combination of Markers is directed to GSTP1 and HINC1. Other sequences of interest include constitutive genes useful as assay controls such as beta-Actin and PTGS2.

Assays for detecting hypermethylation include such techniques as MSP and restriction endonuclease analysis. The promoter region is a particularly noteworthy target for detecting such hypermethylation analysis. Sequence analysis of the promoter region of GSTP1 shows that nearly 72% of the nucleotides are CG and about 10% are CpG dinucleotides.

The invention includes determining the methylation status of certain regions of the Markers in urine or urethral washes and in which the DNA associated with prostate cancer is amplified and detected. Since a decreased level of the protein encoded by the Marker (i.e., less transcription) is often the result of hypermethylation of a particular region such as the promoter, it is desirable to determine whether such regions are hypermethylated. This is seen most demonstrably in the case of the GSTP1 gene and in the panels indicated in the Summary of the Invention. A nucleic acid probe or reporter specific for certain Marker regions is used to detect the presence of methylated regions of the Marker gene. Hypermethylated regions are those that are methylated to a statistically significant greater degree in samples from diseased tissue as compared to normal tissue.

As noted above, urine is the preferred matrix in which the assays of this invention are conducted. Most preferably, it is collected after prostate massage and stored at 4° C. until it can be 30 sedimented. It is most preferably spun down within 4 hours.

Prostatic massage, when conducted in conjunction with the methylation analyses of the invention, is best conducted as follows: the gland is pressed firmly enough to depress the surface from the base to the apex and from the lateral to the median line for each lobe to ensure the release of sufficient number of prostate cells. It is most preferred that this massage procedure is conducted for 20 seconds or less.

Some of the primers/probes or reporter reagents of the invention are used to detect methylation of expression control sequences of the Marker genes. These are nucleic acid sequences that regulate the transcription and, in some cases, translation of the nucleic acid sequence. Thus, expression control sequences can include sequences involved with promoters, enhancers, transcription terminators, start codons (i.e., ATG), splicing signals for introns, maintenance of the correct reading frame of that gene to permit proper translation of the mRNA, and stop codons.

The GSTP1 promoter is the most preferred Marker and is used in conjunction with HICN 1. GSTP1 is a polynucleotide sequence that can direct transcription of the gene to produce a glutathione-s-transferase protein. The promoter region is located upstream, or 5′ to the structural gene. It may include elements which are sufficient to render promoter-dependent gene expression controllable for cell type specific, tissue-specific, or inducible by external signals or agents; such elements may be located in the 5′ or 3′ regions of the of the polynucleotide sequence.

One method of the invention includes contacting a target cell containing a Marker with a reagent that binds to the nucleic acid. The target cell component is a nucleic acid such as DNA extracted from urine by cell lysis and purification (column or solution based) yielding pure DNA that is devoid of proteins. The reagents include components that prime and probe PCR or MSP reactions and detect the target sequence. These reagents can include priming sequences combined with or bonded to their own reporter segments such as those referred to as Scorpion reagents or Scorpion reporters and described in U.S. Pat. Nos. 6,326,145 and 6,270,967. Though they are not the same, the terms “primers” and “priming sequences” may be used in this specification to refer to molecules or portions of molecules that prime the amplification of nucleic acid sequences.

One sensitive method of detecting methylation patterns involves combining the use of methylation-sensitive enzymes and the polymerase chain reaction (PCR). After digestion of DNA with the enzyme, PCR will amplify from primers flanking the restriction site only if DNA cleavage was prevented by methylation. The PCR primers of the invention are designed to target the promoter and transcription region that lies approximately between −71 and +59 bp according to genomic positioning number of M24485 (Genbank) from the transcription start site of GSTP1.

The following examples are provided to illustrate but not limit the invention.

### EXAMPLE 1

### Sample Preparation and MSPCR

### Example (N): Enzymatic Detection of Prostate Cell Methylation Using HIC-1 Marker

Restriction Endonuclease Methylation sensitive qPCR-HIC1

### Background:

Restriction endonuclease HpaII cuts the sequence CCGG but does not cut the methylated form of this sequence, C5mCGG. If the CpG island does not contain C5mCGG, the DNA is cut and no product can be detected after PCR amplification.

### Method:

(1) 300 ng prostate tissue gDNA in 10 ul HpaII endonuclease digestion (37 C/3 hrs, 70 C/10 min)

(2) 2 ul of digested DNA (60 ng) used in qPCR

(3) Restriction endonuclease Hpall specific primer/probes were employed as shown in Table 1.

(3) PCR Reagent used SYBR GreenER qPCR SuperMix Universal (Invitrogen, Cat# 11762-100) or FastStart Taq polymerase (Roche, Cat# 12032929001) for TaqMan probe and Scorpion probes.

(4) The Instruments used to conduct the MSPCR were an ABI 7900 or Cepheid with 2 steps PCR 95 C-15 sec, 60C-60 sec and 40 cycles.

TABLE 1
Primer designs

| | | Seq |
|---|---|---|
| Gene markers | Sequence | ID No. |
| GSTP1-1 | Prostate cancer specific | |
| PCHpaF1 (957) | CGGTCCTCTTCCTGCTGTCT | 1 |
| PCHpaR1 (1244) | CGTACTCACTGGTGGCGAAG | 2 |
| GSTP1-2 | | |
| GSTP_Hpa1005F | TGGGAAAGAGGGAAAGGCTT | 3 |
| GSTP_Hpa1153R | CCCAGTGCTGAGTCACGG | 4 |
| HIC1 | Prostate tissue specific | |
| HIC_Hpa_1022F | TCCCCTGGGAGCTGCGT | 5 |
| HIC_Hpa_1116R | GGGCAGAGCGCGAAGG | 6 |
| TaqMan ® probe | | |
| HIC_Hpa_1056P | CACCTGCTTCCTGCCTCAGCCT | 7 |
| Scorpion probe | | |
| HIC_Hpa_1116R_SCP | FAM-GCCGGCTCCCCGCGCCGCCGG | 8 |
| | C-BHQ1-HEG-GGGCAGAGCGCGAA | |
| | GG | |

### Samples:

(1) Positive control: LnCap cell gDNA from ATCC

(2) Prostrate tissue gDNA from GCI

### Results:

1. Data in table 2 and 3 demonstrate the following:
(a). 5 prostate tissues DNA were discriminated by HIC1 marker from non-prostate DNA (>5 Ct difference).
(b). HIC-1 on contrary shows equivalent performance between 3 cancers and 2 non-cancers of prostate origin.
(c). As results, clear cutoff for both designs of GSTP1 marker was observed between Prostate Cancer and Benign tissue samples after marker normalization with HIC-1(difference between normalized value is >6).
(d) HIC 1 TaqMan® and Scorpion probes detected 60 ng of Hpa II digested LnCap cell DNA (Table 3).

TABLE 2
HIC1 marker performance

| Marker | GSTP1-1 | GSTP1-2 | HIC1 | Normalized by HIC I | |
|---|---|---|---|---|---|
| Positive Control | | | | GSTP1-1 | GSTP1-2 |
| LnCap cell | 24.8 | 23.9 | 23.8 | 1.0 | 0.1 |
| Prostate tissue DNA | | | HIC1 cutoff < 25 | GSTP1 cutoff < 5 | |
| Pca (2b) | 24.8 | 24.5 | 23.7 | 1.1 | 0.8 |
| 141-AC | 25.5 | 24.6 | 22.8 | 2.6 | 1.8 |
| 149-AC | 27.7 | 26.9 | 24.3 | 3.4 | 2.6 |
| 142-BPH | 33.2 | 31.5 | 23.7 | 9.5 | 7.8 |
| P Normal | 33.8 | 34.2 | 22.8 | 10.9 | 11.4 |
| Non-prostate DNA Control | | | | | |
| PBL | 34.0 | 34.7 | 30.7 | | |
| CpGU | 35.5 | 37.4 | 29.5 | | |
| NT | 35.2 | 36.8 | 40.0 | | |

TABLE 3
HIC1 marker performances with TaqMan ®,
Scorpion probe and SYBR Green

| Detection | Probes | Ct |
|---|---|---|
| TaqMan ® probe | HIC_Hpa_1056P | 33.8 |
| Scorpion probe | HIC_Hpa_1116R_SCP | 31.6 |
| SYBR Green | HIC1_SYBR | 28.5 |

### EXAMPLE 2

Seven Scorpion based MSP assays were designed for two different markers (HIC-1 and EDNRB). They were used to screen a prostate tissue panel (N=57) and a bladder-renal tissue panel (N=18). The HIC-1 assays correctly segregated cancer from non-cancer samples based on methylation 100% of the time while the EDNRB was only able to do so 41% of the time in one run of the example. In another run, HIC-1 assays segregated cancer from benign in 44% (HIC-562) and 17% (HIC-119) of the cases based on methylation. Sequences of these assays are listed in Table 3. Likewise, in one run of the HIC-1 assay conducted on urine samples obtained pre and post DRE, the HIC-1 assay correctly identified 100% of cancers (N=13) using post DRE samples while a small but statistically significant number of preDRE samples positive for cancer were not detected. In other runs, pre and post DRE samples were tested with equivalent performance.

TABLE 3
Two designs of nested MSP for HIC-1

| | |
|---|---|
| Sequence | Seq ID No |

| | | | |
|---|---|---|---|
| HIC-S2 (HIC- | Forward primer, | 5′-CGGGTTGGGGTTAGGCG-3′ | 9 |
| 562), Outer | HIC1_553_U17 | | |
| primers | Reverse primer, | 5′-CCGAACGCCTCCATCGTAT-3′ | 10 |
| | HIC1_662_L19 | | |
| HIC-S2 (HIC- | Scorpion, | 5′-FAM-CGGCCGACCGAATAAAAACCGGCCG- | 11 |
| 562), Inner | HIC1_Sc_AS6 | BHQ-HEG-GTTAGGCGGTTAGGGCGTC-3′ | |
| primer-probe | 00_U562 | | |
| set | Reverse primer, | 5′-CCGAACGCCTCCATCGTAT-3′ | 12 |
| | HIC1_662_L19 | | |
| HIC-S4 (HIC- | Forward primer, | 5′-TTTTCGCGTCGGGTTCG-3′ | 13 |
| 119), Outer | HIC1_12_U17 | | |
| primers | Reverse primer, | 5′-ACCGAAAACTATCAACCCTCG-3′ | 14 |
| | HIC1_119_L21 | | |
| HIC-S4 (HIC- | Scorpion, | 5′-FAM-CGGCCGTCGTTCGGGTTTCGGCCG- | 15 |
| 119), Inner | HIC1_Sc_S81_L119 | BHQ-HEG-ACCGAAAACTATCAACCCTCG-3′ | |
| primer-probe | Forward primer, | 5′-GGTTTTCGCGTTTTGTTCGT-3′ | 16 |
| ser | HIC1_28_U20 | | |

### REFERENCES CITED

20020197639

20030022215

20030032026

20030082600

20030087258

20030096289

20030129620

20030148290

20030157510

20030170684

20030194734

20030215842

20030224040

20030232351

20040023279

20040038245

20040048275

20040072197

20040086944

20040101843

20040115663

20040132048

20040137474

20040146866

20040146868

20040152080

20040171118

20040203048

20040241704

20040248090

20040248120

20040265814

20050009059

20050019762

20050026183

20050053937

20050064428

20050069879

20050079527

20050089870

20050130172

20050153296

20050196792

20050208491

20050208538

20050214812

20050233340

20050239101

20050260630

20050266458

20050287553

20070054287

U.S. Pat. No. 4,458,066

U.S. Pat. No. 4,683,195

U.S. Pat. No. 5,242,974

U.S. Pat. No. 5,384,261

U.S. Pat. No. 5,405,783

U.S. Pat. No. 5,412,087

U.S. Pat. No. 5,424,186

U.S. Pat. No. 5,429,807

U.S. Pat. No. 5,436,327

U.S. Pat. No. 5,445,934

U.S. Pat. No. 5,472,672

U.S. Pat. No. 5,527,681

U.S. Pat. No. 5,529,756

U.S. Pat. No. 5,532,128

U.S. Pat. No. 5,545,531

U.S. Pat. No. 5,554,501

U.S. Pat. No. 5,556,752

U.S. Pat. No. 5,561,071

U.S. Pat. No. 5,571,639

U.S. Pat. No. 5,593,839

U.S. Pat. No. 5,599,695

U.S. Pat. No. 5,624,711

U.S. Pat. No. 5,658,734

U.S. Pat. No. 5,700,637

U.S. Pat. No. 5,786,146

U.S. Pat. No. 6,004,755

U.S. Pat. No. 6,136,182

U.S. Pat. No. 6,214,556

U.S. Pat. No. 6,218,114

U.S. Pat. No. 6,218,122

U.S. Pat. No. 6,251,594

U.S. Pat. No. 6,270,967

U.S. Pat. No. 6,271,002

U.S. Pat. No. 6,326,145

U.S. Pat. No. 6,331,393

U.S. Pat. No. 6,335,165

U.S. Pat. No. 6,992,182

Bastian, P et al. (2004) Molecular biomarker in prostate cancer: the role of CpG island hypermethylation Eur Urol 46:698-708

Bastian, P et al. (2007) Prognostic value of CpG island hypermethylation at PTGS2, RAR-beta, EDNRB and other gene loci in patients undergoing radical prostatectomy Eur Urol 51:665-674

Beaucage et al. (1981) Tetrahedron Lett 22:1859-1862

Brookes (1999) The essence of SNPs Gene 234:177-186

Chan, Q et al. (2005) Promoter methylation and differential expression of π-class glutathione S-transferase in endometrial carcinoma J Mol Diagn 7:8-16

Coles et al. (1990) The role of glutathione and glutathione transferases in chemical carcinogenesis CRC Crit Rev Biochem Mol Biol 25:47

Das, R et al. (2006) Computational prediction of methylation status in human genomic sequences PNAS 103:10713-10716

Eckhardt, F et al. (2006) DNA methylation profiling of human chromosomes 6, 20, and 22 Nature Genetics 38:1378-1385

Endoh, M. et al. (2005) RASSF2, a potential tumor suppressor, is silenced by CpG island hypermethylation in gastric cancer Br J Cancer 93:1395-1399

Esteller, M (2005) Aberrant DNA methylation as a cancer-inducing mechanism Ann Rev Pharmacol Toxicol 45:629-656

Esteller, M el al. (2001) A gene hypermethylation profile of human cancer Cancer Res 61:3225-3229

Feng et al, (2005) Detection of hypermethylated genes in women with and without cervical neoplasia J Natl Cancer Inst 97:273-282

Guo, M et al. (2006) Accumulation of promoter methylation suggests epigenetic progression in squamous cell carcinoma of the esophagus Clin Can Res 12:4515-4522

Harden, S et al. (2003) Quantitative GSTP1 Methylation and the Detection of Prostate Adenocarcinoma in Sextant Biopsies J Natl Cancer Inst 95:1634-1637

Jeronimo et al. (2003) Endothelin B receptor gene hypermethylation in prostate adenocarcinoma J Clin Pathol 56:52-55

Jeronimo, C et al. (2001) Quantitation of GSTP1 Methylation in Non-neoplastic Prostatic Tissue and Organ-Confined Prostate Adenocarcinoma J Natl Cancer Inst 93:1747-1752

Jeronimo, C et al, (2004) Quantitative RARbeta2 hypermethylation: a promising prostate cancer marker Clin Cancer Res 10:4010-4014

Kuhn Hoffmann-Berling (1978) CSH-Quantitative Biology 43:63

Lee et al. (1997) CG island methylation changes near the GSTP1 gene in prostatic carcinoma cells detected using the polymerase chain reaction: a new prostate cancer biomarker Cancer Epidemiol 6:443-450

Lee, W et al. (1994) Cytidine methylation of regulatory sequences near the pi-class glutathione S-transferase gene accompanies human prostatic carcinogenesis Proc Natl Acad Sci USA 91:11733-11737

Mannervik et al. (1992) Nomenclature for human glutathione transferases Biochem J 282:305

Markowitz (1952) Portfolio Selection

Nakayama, M et al. (2003) Hypermethylation of the human glutathione S-transferase-π gene

(GSTP1) CpG island is present in a subset of proliferative inflammatory atrophy lesions but not in normal or hyperplastic epithelium of the prostate Am J Pathol 163:923-933

Nakayama, T et al. (2001) The role of epigenetic modifications in retinoic acid receptor beta2 gene expression in human prostate cancers Lab Invest 81:1049-1057

Nelson et al. (1997) Methylation of the 5′ CpG island of the endothelin B receptor gene is common in human prostate cancer Cancer Res 57:35-37

Nojima et al. (2001) CpG hypermethylation of the promoter region inactivates the estrogen receptor-β gene in patients with prostate carcinoma Cancer 9.2:2076-2083

Pao, M et al. (2001) The endothelin receptor B (EDNRB) promoter displays heterogeneous, site specific methylation patterns in normal and tumor cells Hum Molec Genet 10:903-910

Pickett et al. (1989) Glutathione S-transferases: gene structure, regulation, and biological function Annu Rev Biochem 58:743

Radding (1982) Homologous pairing and strand exchange in genetic recombination Ann Rev Genet 16:405-437

Rogers et al. (2006) High concordance of gene methylation in post-digital rectal examination and post-biopsy urine samples for prostate cancer detection J Urol 176:2280-2284

Rushmore et al. (1993) Glutathione S-transferases, structure, regulation, and therapeutic implications J Biol Chem 268:11475

Sasaki et al. (2002) Methylation and inactivation of estrogen, progesterone, and androgen receptors in prostate cancer J Natl Cancer Inst 94:384-390

Satoh et al. (2002) DNA methylation and histone deacetylation associated with silencing DAP kinase gene expression in colorectal and gastric cancers Brit J Cancer 86:1817-1823

Shaw, R et al. (2006) Promoter methylation of p16, RARβ, E-cadherin, cyclin A1 and cytoglobin in oral cancer: quantitative evaluation using pyrosequencing Br J Can 94:561-568

Strunnikova, M et al. (2005) Chromatin inactivation precedes de novo DNA methylation during the progressive epigenetic silencing of the RASSF1A promoter Molec Cellular Biol 25:3923-3933

Toyooka, S et al. (2003) Epigenetic Down-Regulation of Death-associated Protein Kinase in Lung Cancers Clin Cancer Res 9:3034-3041

Warnecke et al. (1997) Detection and measurement of PCR bias in quantitative methylation analysis of bisulphite-treated DNA Nucl Acids Res 25:4422-4426 www.ambion.com/techlib/basics/rnaisol/index.html

Yamanaka et al. (2003) Altered methylation of multiple genes in carcinogenesis of the prostate Int J Cancer 106:382-387

Yan, P et. al, (2003) Differential distribution of DNA methylation within the RASSF1 A CpG island in breast cancer Can Res 63:6178-6186

Yu, J et al (2006) DNA repair pathway profiling and microsatellite instability in colorectal cancer Clin Can Res 12:5104-5111

We claim:

1. A prostate cancer assay having reagents for the detection of the methylation of GSTP1 and HIC-1 in a biological sample.

2. The assay of claim 1 further having reagents for the detection of constitutively expressed gene.

3. The assay of claim 1 wherein the reagent for the detection of the GSTP1 gene is directed to the promoter region.

4. The assay of claim 3 wherein two or more methylation sites are in the promotion region.

5. The assay of claim 3 further comprising instructions including an instruction for normalizing the results based on the detection of the methylation of HIC-1.

6. The assay of claim 1 having endonuclease reagents.

7. The assay of claim 6 wherein the endonuclease cleaves unmethylated HIC-1.

8. The assay of claim 1 further comprising instructions including an instruction to conduct a DRE prior to sample collection.

9. The assay of claim 1 wherein the sample is urine.

10. The assay of claim 1 wherein the sample is prostate tissue.

11. The assay of claim 1 wherein the reagents include Seq. ID No.1 and 5 or 6.

12. A prostate cancer assay having reagents for the detection of methylation sites comprising Seq. ID No. 3 and Seq. ID No. 5 or 6.

13. A kit comprising reagents for detecting methylated nucleotide sequences in GSTP1 and HIC-1 in a biological sample.

14. The kit of claim 13 further comprising nucleotide conversion reagents.

15. The kit of claim 14 wherein the nucleotide conversion reagents include bisulfite.

16. The kit of claim 16 wherein said reagents are designed for use with FFPE tissue samples.

17. The kit of claim 13 wherein the samples are prostate tissue samples.

18. The kit of claim 13 wherein the samples are prostate tissue samples.


SEQUENCE LISTING (16 sequences)

SEQ ID NO: 1 - DNA, 20 bp, human
cggtcctcttcctgctgtct

SEQ ID NO: 2 - DNA, 20 bp, human
cgtactcactggtggcgaag

SEQ ID NO: 3 - DNA, 20 bp, human
tgggaaagagggaaaggctt

SEQ ID NO: 4 - DNA, 18 bp, human
cccagtgctgagtcacgg

SEQ ID NO: 5 - DNA, 17 bp, human
tcccctgggagctgcgt

SEQ ID NO: 6 - DNA, 16 bp, human
gggcagagcgcgaagg

SEQ ID NO: 7 - DNA, 22 bp, human
cacctgcttcctgcctcagcct

SEQ ID NO: 8 - DNA, 38 bp, human
gccggctccccgcgccgccggcgggcagagcgcgaagg

SEQ ID NO: 9 - DNA, 17 bp, human
cgggttggggttaggcg

SEQ ID NO: 10 - DNA, 19 bp, human
ccgaacgcctccatcgtat

SEQ ID NO: 11 - DNA, 44 bp, human
cggccgaccgaataaaaaccggccggttaggcggttagggcgtc

SEQ ID NO: 12 - DNA, 19 bp, human
ccgaacgcctccatcgtat

SEQ ID NO: 13 - DNA, 17 bp, human
ttttcgcgtcgggttcg

SEQ ID NO: 14 - DNA, 21 bp, human
accgaaaactatcaaccctcg

SEQ ID NO: 15 - DNA, 45 bp, human
cggccgtcgttcgggtttcggccgaccgaaaactatcaaccctcg

SEQ ID NO: 16 - DNA, 20 bp, human
ggttttcgcgttttgttcgt